(19) United States
(12) Patent Application Publication
(10) Pub. No.: US 2011/0071226 A1
(43) Pub. Date: Mar. 24, 2011

(54) EMOLLIENT COMPOSITION

(75) Inventors: Pierre Fabre, Castres (FR); Christophe Przybylski, Villefranche De Lauragais (FR); Jean-François Cordoliani, Sainte Foy D'Aigrefeuille (FR); Marion Kopec, Toulouse (FR)

(73) Assignee: PIERRE FABRE DERMO-COSMETIQUE, Boulogne-Billancourt (FR)

(21) Appl. No.: 12/992,808

(22) PCT Filed: May 18, 2009
(86) PCT No.: PCT/EP2009/056021

§ 371 Date: Nov. 15, 2010

(30) Foreign Application Priority Data
May 16, 2008 (FR) 0853187

(51) Int. Cl. A61K 31/047; A61K 31/01; A61P 17/00

(52) U.S. Cl. 514/738; 514/762

(57) ABSTRACT

The invention relates to an emollient composition for topical use that comprises a combination of glycerol, Vaseline and liquid paraffin as an active principle, in the form of an oil-in-water or water-in-oil emulsion. The invention also relates to: the use thereof for preparing a drug for treating dry skin conditions associated with certain dermatoses such as atopic dermatitis, ichthyosis conditions and psoriasis; the use thereof for preparing a drug for treating small superficial burns; and the use thereof for preparing a drug for preventing and/or treating and/or reducing the frequency and intensity of eczema attacks observed in patients suffering from atopic dermatitis.

The invention relates to the field of the treatment of pathologies and conditions associated with a barrier function of affected skin.

The epidermis is a stratified epithelium, of ectodermic origin, in perpetual renewal.

It protects the body from dehydration, mechanical stresses and certain pathogenic attacks.

It is composed of several cell types: more than 90% keratinocytes, but also Langerhans cells, Merkel cells and melanocytes.

Several layers of different morphological nature and cellular composition can be distinguished, from the interior towards the exterior: the basal layer, which is the cellular stratum whose keratinocytes have a capacity of very strong proliferation which ensures self-renewal of the epidermis, then the suprabasal layers (stratum granulosum, stratum spinosum) and finally the stratum corneum (SC).

One of the fundamental functions of the skin is to ensure a barrier between the body and the external medium by opposing in one direction the penetration in the epidermis of fungi, bacteria and allergens from the environment and in the other direction the loss of water.

The quality of the barrier function is evaluated in vivo in man by measurement of insensible water loss and hydration rate and in the mouse by embryonic death by dehydration, cutaneous permeability to a stain or decrease in body weight.

The integrity of the extracellular lipid cement, as well as all the cellular elements of the stratum corneum, and the equilibrium between keratinocyte proliferation and differentiation are essential for the maintenance of a functional epidermal barrier function.

The pH gradient regulates the activities of the various enzymes and thus contributes to the equilibrium of the barrier.

By regulating the secretion of lamellar bodies in the stratum granulosum, calcium ion concentration influences the composition of the extracellular cement of the stratum corneum and thus the equilibrium of the epidermal barrier (Lee et al., Calcium and potassium are important regulators of barrier homeostasis in murine epidermis, J. Clin Invest, 89, 530-538, 1992).

The presence of a water gradient which ranges from 70% in the visible layers of the epidermis to 30% in the inferior layers of the SC and to 15% in the most external cell layers of the epidermis (Warner et al., Electron probe analysis of human skin: determination of the water concentration profile, J. Invest Dermatol, 90, 218-224, 1988) suggests that some water is retained in the junction between the stratum granulosum and the stratum corneum.

One of the functions of water in the stratum corneum is to enable the enzymatic hydrolysis reactions necessary to flexibility of the skin and to normal exfoliation. If the quantity of water present in the SC drops below a critical threshold, enzymatic reactions are disrupted, leading to the adhesion of corneocytes and the accumulation of cells on the surface of the skin. This creates a visible appearance of dryness, and the skin itches, peels and flakes off.

Cutaneous hydration rests on two phenomena: the supply of water by trans-epidermal flow from circulating blood and the retention of epidermal water which brings into play the cutaneous barrier function. However, the barrier with respect to water loss is not absolute. The normal movement of water exchange between the external medium and the internal medium through the stratum corneum is called TEWL (transepidermal water loss) and constitutes part of insensible water loss.

The cutaneous barrier function is affected in most of the skin pathologies that are the most widespread in the population and often accompanied by an inflammatory component (psoriasis, atopic dermatitis, ichtyoses, skin dryness, etc.). It is also affected in a large number of physiological conditions in response to time (skin ageing) or to environmental attacks (UV rays, humidity level, pollution, burns).

Disruption of the barrier function, chronic or acute, makes the body more sensitive to external attacks and dehydration. It can be associated with a disturbance of exfoliation and to hyperproliferation (Jackson et al., Pathobiology of the stratum corneum, West J. Med, 158, 279-285, 1993).

Patent application FR 2 847 467 relates to the use of at least one modulator of the activity of oxysterol 7α-hydroxylase for preparing a cosmetic composition for preventing and/or treating disorders of the skin and/or the mucous membranes affecting the proper functioning of the cutaneous barrier.

Patent application FR 2 831 443 relates to the use of at least one extract of Gingko biloba or Olea europaea, for preparing a composition for improving the barrier function of the skin.

Patent application FR 2 905 857 relates to the use of a composition that comprises an extract of carob pulp for hydrating and/or protecting from skin dryness.

There is a need for a treatment for pathologies and conditions associated with a barrier function of affected skin.

In a surprising and unexpected manner, the Inventors noted that a combination of glycerol, vaseline and liquid paraffin in the form of an oil-in-water or water-in-oil emulsion can treat dry skin conditions.

The Inventors demonstrated that this combination restores a skin barrier to a protective and functional state. They evaluated the hydrating activity of this combination and the subsequent improvement of the skin barrier function by using an ex vivo skin model of induced cutaneous dehydration. Moreover, they observed the expression of molecular epidermal markers potentially involved in homeostasis of the epidermal barrier function by quantitative PCR and immunohistochemistry.

The Inventors also tracked serine protease activity by in situ zymography and the functionality of the skin barrier using fluorescent probes. The results show that the combination of glycerol, vaseline and liquid paraffin in the form of an oil-in-water or water-in-oil emulsion restores serine protease activity and suppresses stress-induced inflammation.

The Inventors also demonstrated that the choice of a particular vaseline in this combination is particularly advantageous to achieve the results above. Vaseline as an occlusive agent and emollient is particularly important in the composition. Indeed, by forming a protective film on the skin, it helps compensate for the deficiency of the affected barrier function. The quality of film formed on the skin very strongly depends on the rheological properties of the vaseline used in manufacture.

The present invention thus has as an aim a composition for topical use that comprises a combination of glycerol, vaseline and liquid paraffin as an active ingredient, in the form of an oil-in-water or water-in-oil emulsion.

In the context of the present invention, “active combination” means a combination of glycerol, vaseline and liquid paraffin, in the form of an oil-in-water or water-in-oil emulsion.

Advantageously, the glycerol, the vaseline and the liquid paraffin possess the criteria described and regulated according to the “European Pharmacopeia”, 6th Edition.

Advantageously, the vaseline of the active combination has a drop point between 35° C. and 70° C., preferably between 51° C. and 57° C., and in a particularly preferred manner approximately 54° C. The drop point is measured according to process 2.2.17 described in the “European Pharmacopeia”, 6th Edition.

Advantageously, the vaseline of the active combination has a consistency between 175 1/10 mm and 195 1/10 mm, preferably approximately 185 1/10 mm (cone penetration at 25° C.)

Advantageously, the vaseline of the active combination has a viscosity between 4 cSt and 5 cSt at 100° C., preferably approximately 4.8 cSt at 100° C.

Advantageously, the vaseline of the active combination has a 500 MHz NMR spectroscopy spectrum of carbon-13 (¹³C) that comprises a peak at 24.55 ppm whose area relative to a 1% tetramethylsilane (TMS) control is between 4 and 8.

In the inventive composition, the active combination is present in a proportion between 10% and 50% and preferentially between 20% and 30% by weight compared to the total weight of the composition; the glycerol concentration is between 5% and 30%, preferentially between 10% and 20% and in a particularly preferred manner approximately 15% by weight compared to the total weight of the composition; the concentration of vaseline is between 3% and 20%, preferentially between 5% and 10% and in a particularly preferred way approximately 8% by weight compared to the total weight of the composition; and the concentration of liquid paraffin is between 0.5% and 5%, preferentially between 1% and 3% and in a particularly preferred way approximately 2% by weight compared to the total weight of the composition.

In the aqueous phase, water is between 30% and 80% by weight compared to the total weight of the composition.

Advantageously, the inventive composition consists of approximately 15% glycerol, approximately 8% vaseline and approximately 2% liquid paraffin by weight compared to the total weight of the composition.

The dermatological composition according to the invention further comprises typical dermatologically compatible excipients.

The dermatological composition according to the present invention can be prepared in the form of a water-in-oil (W/O) or oil-in-water (O/W) emulsion, a multiple emulsion such as, for example, a water-in-oil-in-water (W/O/W) or an oil-in-water-in-oil (O/W/O) emulsion, or in the form of a hydrodispersion or a lipodispersion, a gel or an aerosol.

The dermatologically compatible excipients can be any excipient among those known to the person skilled in the art in order to obtain a composition for topical application in the form of a cream, lotion, gel, pomade, emulsion, microemulsion, spray, etc.

The inventive composition can in particular contain additives and formulation aids, such as emulsifiers, thickeners, gelling agents, water fixing agents, spreading agents, stabilizers, dyes, perfumes and preservatives.

Suitable emulsifiers include stearic acid, trolamine and PEG-40-stearate.

Preferably, the inventive composition has approximately 5% emulsifier by weight compared to the total weight of the composition.

Advantageously, the inventive composition has between 1% and 5% stearic acid, preferably approximately 3% by weight compared to the total weight of the composition.

Advantageously, the inventive composition has between 0% and 2% trolamine, preferably approximately 0.5% by weight compared to the total weight of the composition.

Advantageously, the inventive composition has between 0% and 2% PEG-40-stearate, preferably approximately 0.5% by weight compared to the total weight of the composition.

Suitable thickeners include glycerol monostearate and PEG 600.

Preferably, the inventive composition has approximately 5% thickeners by weight compared to the total weight of the composition.

Advantageously, the inventive composition has between 2% and 10% glycerol monostearate, preferably approximately 5% by weight compared to the total weight of the composition.

Advantageously, the inventive composition has between 2% and 10% PEG 600, preferably approximately 5% by weight compared to the total weight of the composition.

Suitable preservatives include propyl parahydroxybenzoate and chlorocresol.

Preferably, the inventive composition has approximately 0.1% preservatives by weight compared to the total weight of the composition.

Advantageously, the inventive composition has between 0.05% and 1% propyl parahydroxybenzoate, preferably approximately 0.1% by weight compared to the total weight of the composition.

Suitable spreading agents include dimethicone and polydimethylcyclosiloxane.

Preferably, the inventive composition has approximately 2% spreading agents by weight compared to the total weight of the composition.

Advantageously, the inventive composition has between 0.2% and 2% dimethicone, preferably approximately 0.5% by weight compared to the total weight of the composition.

Advantageously, the inventive composition has between 1% and 3% polydimethylcyclosiloxane, preferably approximately 2.5% by weight compared to the total weight of the composition.

Suitable water fixing agents include polyethylene glycol, preferably polyethylene glycol 600.

Preferably, the inventive composition has approximately 8% water fixing agents by weight compared to the total weight of the composition.

Advantageously, the inventive composition has between 2% and 10% polyethylene glycol, preferably approximately 5% by weight compared to the total weight of the composition.

The water used for the aqueous phase of the emulsion can be distilled or thermal water that possesses dermato-cosmetic properties.

Advantageously, the inventive composition consists of:
approximately 15% glycerol,
approximately 8% vaseline,
approximately 2% liquid paraffin, and as excipients:
approximately 1% to 5% stearic acid,
approximately 2% to 10% glycerol monostearate,
approximately 1% to 3% polydimethylcyclosiloxane,
approximately 0.2% to 2% dimethicone,
approximately 2% to 10% polyethylene glycol 600,
approximately 0% to 2% trolamine,
approximately 0.05% to 1% propyl parahydroxybenzoate,
up to 100% with water.

The present invention also has as an aim the use of a composition according to the invention for preparing a drug for treating dry skin conditions associated with certain dermatoses such as atopic dermatitis, ichthyosis conditions and psoriasis.

The present invention also has as an aim the use of a composition according to the invention for preparing a drug for treating small superficial burns.

The present invention also has as an aim the use of a composition according to the invention for preparing a drug for preventing and/or treating and/or reducing the frequency and intensity of eczema attacks observed among patients suffering from atopic dermatitis.

The present invention is illustrated by the following examples.

### FIGURES

FIG. 1: 500 MHz NMR spectrum of carbon-13 for a 5 g sample of Syntadex A vaseline (Synthéal) and of composition A.

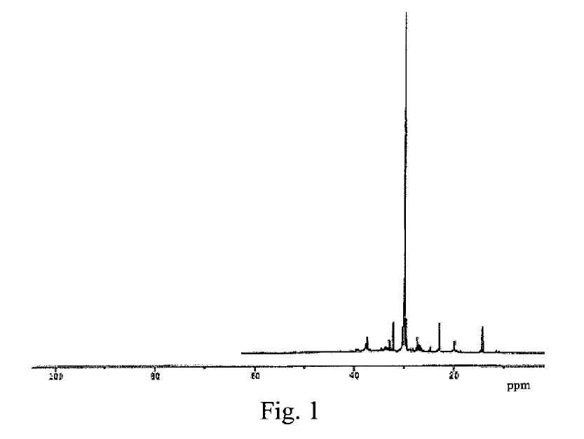

### EXAMPLES

### Example 1

### Formulations

Composition A
15 g glycerol,
8 g vaseline,
2 g liquid paraffin,
0.5 g trolamine,
and as excipients: stearic acid, glycerol monostearate, polydimethylcyclosiloxane, dimethicone, polyethylene glycol (PEG) 600, propyl parahydroxybenzoate,
water up to 100 g.

Composition A′
15 g glycerol,
8 g vaseline,
2 g liquid paraffin,
1.5 g stearic acid,
5 g glycerol monostearate,
1.5 g polydimethylcyclosiloxane,
0.5 g dimethicone,
5 g polyethylene glycol 600,
0.15 g trolamine,
0.1 g propyl parahydroxybenzoate,
water up to 100 g.

Composition B
15 g glycerol,
8 g vaseline,
2 g liquid paraffin,
0.5 g PEG-40-stearate,
and as excipients: stearic acid, glycerol monostearate, polydimethylcyclosiloxane, dimethicone, polyethylene glycol 600, chlorocresol,
water up to 100 g.

Composition B′
15 g glycerol,
8 g vaseline,
2 g liquid paraffin,
3 g stearic acid,
5 g glycerol monostearate,
2 g polydimethylcyclosiloxane,
0.5 g dimethicone,
0.1 g trolamine,
3 g polyethylene glycol 600,
0.5 g PEG-40-stearate,
0.075 g chlorocresol,
water up to 100 g.

### Example 2

### Analysis of the Regulation of Induced Cutaneous Dehydration

Here, we evaluate the hydrating activity of composition A and the subsequent improvement of the skin barrier function by using an ex vivo skin model of induced cutaneous dehydration.

We observe expression of the differential molecular epidermal markers by quantitative PCR and immunohistochemistry.

We also track the activity of serine protease enzymes by in situ zymography and the degradation of corneodesmosomal proteins by Western blotting.

The functionality of the skin barrier is analysed by using fluorescent probes.

Materials and Methods

I. Tissue Models

1. Preparation of Cutaneous Explants

The laboratory recovers skin samples from the operational waste of plastic surgery (mammary reductions). The use of these samples falls within the scope of “the declaration of activity of preservation and preparation of elements of the human body for the needs of the research programme of the group Pierre Fabre” made to the French Ministry for Higher Education and Research.

These samples are washed in 10 baths of PBS and then 2 cm diameter discs are cut out with a punch. The cutaneous explants are spread out on a grid in a Petri dish and a 1 cm diameter ring is embedded in the skin to delimit the treatment area.

2. Kinetics of the Models

For the induced dehydration model, the skin is desiccated for 2 hours under the cell culture hood in an uncovered dish and then is put in the incubator for a topical treatment with or without the active combination for 2 hours. The dehydration stress negative control undergoes the same kinetics in a closed Petri dish.

3. Samples for Analysis

After the treatment, 2 biopsies of 6 mm diameter are taken for analysis of RNA expression and a biopsy of 4 mm diameter is enclosed in a block of Tissue Tek® resin (Sakura Finetek) for histology. For the analysis of proteins, the skin is exposed to a thermal shock in a 60° C. water bath for 5 minutes then a 4° C. bath for 2 minutes in order to separate the epidermis from the dermis.

The biopsies and the epidermises are frozen in liquid nitrogen and stored at −80° C. before being analysed.

II. Transcriptome Analysis by Quantitative PCR

The skin biopsies are crushed in a mortar pre-cooled with liquid nitrogen and RNAs are extracted using an RNeasy® kit (QIAGEN) according to the manufacturer's recommendations. The RNA is then assayed using a Bioanalyser 2100® (Agilent Technologies) on RNA 6000 Nano LabChip® chips. cDNA is obtained from 1 μg RNA by reverse transcription enzymatic reaction performed with an Access RT-PCR Core Reagents® kit (Promega), using oligo dT primers. Gene expression levels are analysed by quantitative PCR on an iCycler iQ® (Biorad) fluorescence thermal cycler with PCR iQ™SYBR® Super Green Mix kits (Biorad) according to a protocol of 40 cycles comprising denaturation at 95° C. (15 sec) and extension at 60° C. (1 min). The accumulation of the PCR product proportional to the fluorescence emission (intercalating SYBR®Green) is visualized cycle after cycle using the iCycler software.

The iCycler version 3.1 analysis software delivers raw values of C_{T} (cycle threshold), the cycle from which cDNA amplification begins. The expression of several reference genes is analysed in parallel using the program Genorm version 3.4, which makes it possible to choose the most stable reference gene from one sample to the next. This gene is then used as the reference to normalise the results by the calculation ΔC_{T}=(C_{T} gene of interest)−(C_{T} reference gene).

The induction factor (IF) is then calculated for each treatment compared to the corresponding control condition. IF=2^{-ΔΔC}^{_{T}} where ΔΔC_{T}=(treated ΔCT)−(control ΔCT). mRNA expression is evaluated in duplicate for five experiments arising from 5 different individuals. When the induction factor compared to the control is greater than 2, gene expression is considered to be induced and when it is less than 0.5, expression is considered to be repressed. The effect of the active principle on the response to stress caused in the model is evaluated by the percentage of inhibition calculated with the following formula:

(% inhibition to the stress response)=100*(((stressed IF)−(control without stress IF))−((treated IF)−(control without stress IF)))/((stressed IF)−(control without stress IF)).

Compared to the model of the study, the “control without stress” condition corresponds to the not desiccated control; the “stressed” condition corresponds to a skin biopsy which was desiccated for 2 hours and then which spent 2 additional hours in the control condition (i.e., without topical treatment); finally, the “treated” condition is the skin which underwent 2 hours of drying followed by 2 hours of topical treatment by emollient.

III. Protein Expression Analysis by Western Blot

The treated epidermises are crushed in a mortar cooled with liquid nitrogen and the proteins are extracted in a RIPA lysis buffer (50 mM Tris-HCl pH 8; 150 mM NaCl; 1% Triton X-100; 1% Na⁺-deoxycholate; 0.1% SDS; 5 mM EDTA; 100 mM DTT; protease inhibiter cocktail (P8340, SIGMA).

The proteins are then assayed by the DC-DC Protein Assay (Biorad) method and analysed by Western blot. For each condition, 25 μg to 40 μg of total proteins are deposited on 7.5% polyacrylamide Tris-Glycine gels. The protein mixture is separated by electrophoresis using the Mini Protean II system (Biorad) and the proteins are transferred to a PVDF membrane (Hybond-P, Amersham). The protein of interest is revealed by a specific antibody and an ECL+ kit (Amersham). The quantity of proteins and the proportion of degraded form are calculated using the Image Master TotalLab version 1.11 software (Amersham) after normalisation compared to β-actin (reference protein).

IV. Histological Techniques

The skin biopsies are sectioned with the cryotome (Leica CM 3050s) in 5 μm thick sections and are deposited on observation slides (Starfrost®).

1. Immunohistochemistry

The cryosections are fixed for 10 minutes in acetone at 20° C. and then rehydrated in PBS before being analysed by immunochemical labelling. After fixing and rehydration, the skin sections are saturated with a 3% BSA solution and incubated for 1 hour with the primary antibody directed against the protein of interest. Next, they are incubated for 1 hour with the secondary antibody coupled to an Alexa-488 or Alexa-555 fluorochrome and finally mounted in Mowiol containing DAPI to stain the nuclei.

2. In Situ Zymography

After fixing for 10 minutes in acetone at −20° C., the sections are rinsed in a washing solution (1% Tween 20 in water) and are incubated for 2 hours at 37° C. with a solution containing the specific substrate of the enzymes of interest coupled to a fluorophore (secondary). When the enzyme is active, the fluorophore is cleaved, releasing a fluorescent signal observable under the microscope. The labelled slides are then observed under the epifluorescence microscope (Nikon Eclipse 50i) or under the Zeiss Axiovert 100 inverted confocal microscope.

3. Fluorescent Probe

After the dehydration treatment, the cutaneous explants are incubated for one additional hour in the incubator at 37° C. with 1 mM Lucifer yellow carbohydrazide lithium salt fluorescent probe (Invitrogen) in HBSS buffer. The skin is then rinsed in a HBSS bath for 1 minute and then 4 mm diameter biopsies are taken and enclosed in Tissue Tek® resin (Sakura Finetek) (Matsuki et al., 1998). The skin is then sectioned, the nuclei are stained with DAPI and the slides are observed under the fluorescence microscope at a wavelength of 450 nm as described above.

Results

I. Model of Barrier Function Rupture by Induced Drying

1. Measurement of Cutaneous Permeability by a Fluorescent Probe

The first analysis consisted in studying a fundamental functional parameter in the cutaneous barrier function: permeability of the superior layers of the epidermis. Incubation of the skin with a fluorescent probe (Lucifer yellow) after the drying experiment made it possible to characterise the modulation of cutaneous permeability. In the control condition, labelling is very weak and superficial; the probe penetrates little through the stratum corneum and is eliminated during rinsing. After two hours of drying, labelling is observable in the deeper layers of the stratum corneum. Drying makes the skin more permeable; its barrier function is deteriorated. Topical treatment by composition A following the two hours of drying restores the impermeability of the SC with respect to the probe; labelling is again weak and superficial, as under the control condition. It can then be concluded that the hydrating treatment has a repairing effect on the desiccated skin and on the cutaneous barrier function observable on the tissue model.

2. Effects on the Regulation of the Transcriptome and the Proteome

The expression of various genes potentially involved in the homeostasis of the epidermal barrier function was measured by quantitative PCR under the various stress or treatment conditions of the drying model. The analysis by immunohistochemistry showed the reorganisation of the expression of certain proteins in terms of localisation, for example the tight junctions. The degradation of corneodesmosomal proteins was analysed by Western blot.

The targets studied using these various approaches were grouped according to their physiological role (see Table 1). The objective of this study is to observe a response to visualisable stress and a correction of the effect of stress by the topical application of composition A.

The work performed also demonstrated the various levels of regulation of certain targets. It thus is noted that exfoliation enzymes were not regulated on the transcriptional level but more particularly on the level of their activity (see Results 3).

TABLE 1
Summary of targets and the
pharmacological response studied in the
drying model.

| | | Inhibition of the |
|---|---|---|
| | Response to | response to stress |
| Tools | stress | by composition A |
| Cutaneous | Y | Y |
| permeability | | |
| Desmoglein 1 | Y | Y |
| CDSN | N | N |
| Desmocollin | Y | Y |
| Plakoglobin | Y | N |
| KLK5 | Y | N |
| KLK7 | N | N |
| KLK8 | N | N |
| Serine proteases | Y | Y |
| Cathepsin D | N | N |
| ABC A12 | Y | Y |
| ABC G1 | N | N |
| B-GC | N | N |
| DES 2 | N | N |
| Filaggrin | Y | Y |
| Involucrin | N | N |
| TG1 | N | N |
| TG3 | N | N |
| Caspase 14 | N | N |
| Elox-3 | Y | N |
| 12R-LOX | N | N |
| HAS2 | Y | N |
| CD44 | N | N |
| AQP3 | Y | Y |
| NHE1 | Y | Y |
| IL-1α | Y | Y |
| Occludin | Y | Y |
| Claudin 1 | Y | Y |
| Claudin 4 | Y | Y |
| ZO-1 | N | N |
| E-cadherin | N | N |
| β-catenin | N | N |

Y = Yes, the target reacts in the model;
N = No, the target does not react in the model.

3. Measurement of Enzymatic Activity Related to Exfoliation

Serine protease activity was evaluated by in situ zymography on the dehydration model and observed under the confocal microscope in the control condition after two hours of drying and after two hours of drying followed by two hours of incubation with composition A. Labelling is most intense under the control condition; it corresponds to normal strong activity. This labelling decreases and becomes irregular along the stratum corneum after two hours of drying whereas its intensity is increased and localization of the activity reorganised after two hours of incubation with composition A. The effect of drying is to decrease and disrupt enzymatic activity. These results are consistent with the decrease in the degradation of corneodesmosomal proteins which is observed with drying and confirms the effect of drying on the decrease in exfoliation observed on the model developed. Composition A is able to restore the enzymatic activity of dehydrated skin, which confirms the effect of this composition on a return to exfoliation homeostasis.

These results show that composition A restores the expression level of molecular targets whose expression is increased by the stress of induced cutaneous dehydration. Composition A also restores serine protease activity. Further, the topical application of composition A eliminates stress-induced inflammation.

The whole of these results suggest that topical application of composition A restores the skin barrier function, limits

### Example 3

### Characterization by NMR of Syntadex A Vaseline

5 g of the sample are solubilised in deuterated chloroform for measurement by 500 MHz NMR of carbon-13.

Syntadex A vaseline (Synthéal) exhibits a characteristic 500 MHz NMR spectroscopy spectrum of carbon-13, notably comprising a peak at 24.55 ppm whose area relative to a 1% tetramethylsilane (TMS) control is between 4 and 8.

This same peak is found in composition A.

1.-14. (canceled)

15. A composition for topical use that comprises a combination of glycerol, vaseline and liquid paraffin as an active ingredient, in the form of an oil-in-water or water-in-oil emulsion.

16. The composition of claim 15 in which the vaseline has a 500 MHz NMR spectroscopy spectrum of carbon-13 that comprises a peak at 24.55 ppm whose area relative to a 1% tetramethylsilane (TMS) control is between 4 and 8.

17. The composition of claim 15 in which the vaseline has a dropping point between 51° C. and 57° C., in particular 54° C.

18. The composition of claim 15 in which the vaseline has a consistency between 175 1/10 mm and 195 1/10 mm (cone penetration at 25° C.).

19. The composition of claim 18 in which the vaseline has a consistency of approximately 185 1/10 mm (cone penetration at 25° C.).

20. The composition of claim 15 in which the vaseline has a viscosity between 4 cSt and 5 cSt at 100° C.

21. The composition of claim 20 in which the vaseline has a viscosity of approximately 4.8 cSt at 100° C.

22. The composition of claim 15 that comprises approximately 15% glycerol, approximately 8% vaseline and approximately 2% liquid paraffin.

23. The composition of claim 15 that comprises one or more excipients selected from the group comprised of stearic acid, glycerol monostearate, polydimethylcyclosiloxane, dimethicone, polyethylene glycol 600, trolamine, propyl parahydroxybenzoate, chlorocresol, PEG-40-stearate, distilled water.

24. A method of treatment of the conditions of cutaneous dryness of certain dermatoses such as atopic dermatitis, ichthyosis conditions and psoriasis comprising the topical administration of the composition of claim 15 to a patient in need thereof.

25. A method of treatment of small superficial burns comprising the topical administration of the composition of claim 15 to a patient in need thereof.

26. A method of prevention and/or treatment and/or decrease of the frequency and the intensity of the eczema attacks comprising the topical administration of the composition of claim 15 to a patient suffering from atopic dermatitis in need thereof.

27. Use of a vaseline with a dropping point between 51° C. and 57° C. for preparing a composition for topical use that comprises a combination of glycerol, vaseline and liquid paraffin as an active ingredient, in the form of an oil-in-water or water-in-oil emulsion.

28. Use of a vaseline with a 500 MHz NMR spectroscopy spectrum of carbon-13 that comprises a peak at 24.55 ppm whose area relative to a 1% tetramethylsilane (TMS) control is between 4 and 8 for preparing a composition for topical use that comprises a combination of glycerol, vaseline and liquid paraffin as an active ingredient, in the form of an oil-in-water or water-in-oil emulsion.
